(19) United States
(12) Patent Application Publication
(10) Pub. No.: US 2020/0001026 A1
(43) Pub. Date: Jan. 2, 2020

(54) SMART SPACER FOR AN INHALER

(71) Applicant: Mylan Inc., Canonsburg, PA (US)

(72) Inventors: Eric W. Starr, Butler, PA (US); Molly Knewtson, Pittsburgh, PA (US); Benjamin Che, Pittsburgh, PA (US)

(21) Appl. No.: 16/021,690

(22) Filed: Jun. 28, 2018

(51) Int. Cl. A61M 15/00

(52) U.S. Cl. CPC A61M 15/009; A61M 2205/3334; A61M 2230/42; A61M 2205/3553; A61M 2205/3584; A61M 2205/587; A61M 15/008; A61M 2205/50

(57) ABSTRACT

A spacer is provided comprising a chamber having an interior surface and an exterior surface. The interior surface is configured to receive aerosolized medicament from an inhaler and has at least one sensor for measuring airflow in the chamber. The spacer includes an actuator disposed on the exterior surface configured to initiate measurement of airflow in the chamber. Methods of making and using a spacer are also provided.

### CROSS REFERENCE TO RELATED APPLICATIONS

This application claims priority to U.S. Provisional Application Ser. No. 62/526,555 filed on Jun. 29, 2017, the entire contents of which is incorporated herein by reference in its entirety.

### BACKGROUND

Patients with respiratory disorders, such as asthma and COPD, are commonly prescribed medicaments that are self-administered via the use of inhaler devices such as, for example, metered-dose inhalers (MDIs) and soft mist inhalers (SMI). Inhalers generate a cloud of aerosolized medicament which a patient inhales through their mouth in order to deliver the medication to their airways and lungs. Often, a patient will improperly use an inhaler which can cause improper dosing of the medicament.

One way to reduce improper use of an inhaler is through the use of an add-on device, such as a spacer. A spacer is a device that is paired with an inhaler such that the spacer suspends the aerosolized medicament within the spacer chamber, allowing the patient to inhale the full dose of the medicament with one or more deep breaths. However, even with the availability of a spacer, many patients have difficulty managing their respiratory disorder for several reasons, including inconsistent, under, or over utilization of the medicament, ineffective inhaler technique, and/or infrequent assessment of and feedback of their lung health.

Some spacers include patient technique coaching via mechanical means such as a whistle for when the patient inhales too rapidly. However, these mechanical means do not generate longitudinal trends nor do they provide information on patient inhalation volume or duration.

One way patient's test their lung function and, thus, assess their lung function is by a spirometer. A spirometer is a separate device from the inhaler, where the patient can measure their inhalation volume or duration. Spirometers have the disadvantage in that the patient would have to carry the spirometer separately from the spacer. Further, the spirometer cannot be chronologically associated with patient inhaler technique or medicament utilization.

Therefore, it would be beneficial to provide a smart spacer that measures and records a patient's inhaled airflow and exhaled airflow. It would also be beneficial to provide a smart spacer that can generate metrics on inhalation technique, record medicament utilization and generate metrics on lung health so that a practitioner monitoring a patient's respiratory disorder can track a patient's improvement. Further, it would be beneficial to provide a smart spacer that offers a spirometry feature.

### SUMMARY

The present application provides a smart spacer that measures and records a patient's inhaled airflow and exhaled airflow. The smart spacer also generates in real time metrics on inhalation technique, records medicament utilization and generates metrics on lung health in real time so that a patient can track their own improvement, as well as, a practitioner monitoring a patient's respiratory disorder can track said patient's improvement. Further, these metrics can be displayed on the patient's phone, the practitioner's computer or other display. The smart spacer provides a mode for initiating a spirometry feature that can be transmitted to the patient's phone and transmitted to the practitioner to monitor patient therapy and adjust the dose of the aerosolized medicament being administered to the patient.

In one embodiment, a spacer is provided. The spacer comprises a chamber having an interior surface and an exterior surface. The interior surface of the chamber is configured to receive aerosolized medicament from an inhaler and has at least one sensor for measuring airflow in the chamber. The spacer includes an actuator that is disposed on the exterior surface of the chamber that is configured to initiate measurement of airflow in the chamber.

In another embodiment, a spacer is provided. The spacer comprises a chamber having an interior surface and an exterior surface. The interior surface of the chamber is configured to receive inhalation air and exhalation air from a patient. The interior surface has at least one sensor for measuring airflow in the chamber. The spacer includes an actuator that is disposed on the exterior surface of the chamber that is configured to initiate measurement of airflow in the chamber.

In yet another embodiment, a method of making a spacer is provided. The method comprises providing a spacer comprising a chamber having an interior surface, an exterior surface, and a proximal opening and a distal opening opposite the proximal opening, the interior surface of the chamber configured to receive inhalation air and exhalation air from a patient, attaching to the interior surface of the chamber at least one sensor for measuring airflow in the chamber; and attaching an actuator on the exterior surface of the chamber, the actuator configured to initiate measurement of airflow in the chamber.

In one embodiment, a method of using a spacer is provided. The method comprises attaching an inhaler to a distal end opening of the spacer, the spacer comprising a chamber having an interior surface and an exterior surface, the interior surface of the chamber configured to receive aerosolized medicament from an inhaler and having at least one sensor for measuring airflow in the chamber; and an actuator disposed on the exterior surface of the chamber configured to initiate measurement of airflow in the chamber.

In some embodiments, a method of using a spacer is provided. The method comprises removing an inhaler from a distal end opening of the spacer, the spacer having a chamber having an interior surface and an exterior surface, the interior surface of the chamber configured to receive inhalation air and exhalation air from a patient, the interior surface having at least one sensor for measuring airflow in the chamber; and an actuator disposed on the exterior surface of the chamber configured to initiate measurement of airflow in the chamber.

Other features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE FIGURES

In part, other aspects, features, benefits and advantages of the embodiments will be apparent with regard to the following description, appended claims, and accompanying drawings in which:

FIG. 1 illustrates a side view of a smart spacer engaged with an inhaler. The smart spacer comprises a chamber having an interior surface and an exterior surface. The interior surface of the chamber is configured to receive aerosolized medicament from the inhaler and has at least one sensor for measuring airflow in the chamber. The spacer includes an actuator that is disposed on the exterior surface of the chamber that is configured to initiate measurement of airflow in the chamber. The smart spacer shown includes a microcontroller and indicia, such as light emitting diodes (LED) disposed on the exterior surface of the chamber.

FIG. 2 illustrates a perspective view of the smart spacer of FIG. 1 and an inhaler. The inhaler will engage the smart spacer at a distal end opening of the chamber of the smart spacer.

FIG. 3 illustrates a side view of the smart spacer of FIG. 1 engaged with an inhaler. A patient is shown operating the smart spacer. The patient places their mouth on a proximal end opening of the spacer. The patient presses an actuator on the spacer shown as a button. The inhaler is actuated by the patient and the medicament is then released from the inhaler into the chamber of the smart spacer. The medicament is then inhaled by the patient. Measurement of inhalation airflow in the interior of the chamber is measured by the at least one sensor while the patient inhales the medicament.

FIG. 3A illustrates an enlarged perspective view of the interior surface of the chamber defining a recess or indent that engages the at least one sensor for a friction fit or a snap fit engagement. In this way, if needed, the at least one sensor can be removed during washing of the spacer or the at least one sensor.

FIG. 4 illustrates the smart spacer of FIG. 1 paired with a computer, such as a smartphone via a wireless medium, such as a Bluetooth® radio.

FIG. 5 illustrates the smart spacer of FIG. 1. The smart spacer includes a Bluetooth® radio for wireless connection. The Bluetooth® radio is configured to pair with a Bluetooth® radio of a computer, such as a smartphone. The Bluetooth® radio transmits data compiled from airflow measurements to the smartphone. The smartphone or other computers is loaded with a software program that stores the data and interfaces with the patient such that the data can be shared with the patient or practitioner. The data can then be transmitted from the smartphone via Wi-Fi to a web dashboard on a second computer or a cloud network.

FIG. 6 is a schematic of various inputs and outputs related to inhaler use.

FIG. 7 is a flow chart illustrating inhaler usage and feedback loops of the spacer in some embodiments, when the spacer is in the inhalation mode.

FIG. 8 is a flow chart illustrating inhaler usage and feedback loops of the spacer in some embodiments, when the spacer is in the inhalation mode.

FIG. 9 is a flow chart illustrating inhaler usage and feedback loops of the spacer in some embodiments, when the spacer is in the exhalation mode.

FIG. 10 is a flow chart of the spacer in the inhalation mode for medicament utilization tracking. For medicament utilization tracking, a medicament utilization event is logged whenever the patient presses on the actuator on the spacer and inhalation is recorded by the spacer.

FIG. 11 is a flow chart of the spacer in an inhalation mode. In the inhalation mode, the spacer is used to track the inhalation technique of the patient such that when the at least one sensor detects the direction of airflow, volumetric flow rate is measured throughout the duration of inhalation. From data compiled, the spacer calculates the total inhaled volume and duration which is then used guide the patient on inhalation technique through an application of a personal computer, such as a smartphone. The practitioner can also view these results via a web dashboard. Medicament utilization tracking is a part of the inhalation mode.

FIG. 12 is a flow chart of the spacer exhalation mode or spirometry mode. In the exhalation mode, the spacer acts as a spirometer. If the at least one sensor detects via the direction of airflow that the patient has begun exhaling, then the volumetric airflow rate is measured throughout the duration of the exhalation. From data compiled, the spacer calculates lung health metrics such as forced expiratory volume within the first second of exhalation (FEV1) and peak expiratory flow (PEF). The results are then displayed on a longitudinal graph with interpreted metrics through an application of a personal computer, such as a smartphone. The practitioner can also view these results via a web dashboard.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

The present disclosure may be understood more readily by reference to the following detailed description of the disclosure presented in connection with the accompanying drawings, which together form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. The following description is presented to enable any person skilled in the art to make and use the present disclosure.

Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

### Definitions

As used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, or the like, are also used to describe various elements, regions, sections, etc. and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features.

The term “spacer” refers to a device used to increase the ease of administering aerosolized medication from an inhaler to a patient. A spacer adds space in the form of a tube or chamber between the mouth and canister of medication. A spacer assists a patient to breathe deeply, as well as assist patients that are unable to synchronize their breathing so that they inhale just as the inhaler is actuated.

The term “spirometer” refers to a device for measuring the volume of air inspired and expired by the lungs. A spirometer measures ventilation, the movement of air into and out of the lungs.

The term “metered dose inhaler” (MDI) refers to a device that delivers a specific amount of medicament to the lungs in the form of a short burst of aerosolized medicament that is usually self-administered by the patient via inhalation.

The term “soft mist inhaler (SMI)” can include a multidose, propellant-free, hand-held liquid inhaler that represents a category of inhaler devices. “Soft Mist” is used to describe the mechanism of aerosol generation and the qualities of the aerosol cloud. An example of a SMI is Respimat® (Boehringer Ingelheim, Ingelheim am Rhein, Germany).

The term “medicament” includes a substance suitable for oral or nasal inhalation. The medicament can include at least one active pharmaceutical ingredient and an excipient.

The term “pharmaceutically acceptable salt” or “salt” comprises inorganic and organic salts. Examples of organic salts may include formate, acetate, trifluoroacetate, propionate, butyrate, lactate, citrate, tartrate, malate, maleate, succinate, methanesulfonate, benzenesulfonate, xinafoate, pamoate, and benzoate. Examples of inorganic salts may include fluoride chloride, bromide, iodide, phosphate, nitrate and sulfate.

As used herein, the term “medicament” and “active pharmaceutical ingredient” (API) as used herein, are interchangeable terms and include any substance (i.e., compound or composition of matter) which, when administered to an organism (human or animal) induces a desired pharmacologic and/or physiologic effect by local and/or systemic action. The term therefore encompasses substances traditionally regarded as actives, drugs or bioactive agents, as well as biopharmaceuticals typically employed to treat a number of conditions which is defined broadly to encompass diseases, disorders, infections, or the like. Exemplary medicaments (APIs) include, without limitation, antibiotics, antivirals, H₂-receptor antagonists, 5HT₁ agonists, 5HT₃ antagonists, COX2-inhibitors, steroids (e.g., prednisone, prednisolone, dexamethasone), non-steroidal anti-inflammatory medicaments (APIs), muscarinic M3 receptor agonists or anticholinergic agents, β2-adrenoceptor agonists, compounds having a dual muscarinic antagonist and β2-agonist activity and glucocorticoid receptor agonists.

In some embodiments, the medicament (API) is ipratropium, tiotropium, oxitropium, trospium, aclidiniums, perenzepine, telenzepine, ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, prednisone, prednisolone, flunisolide, triamcinolone acetonide, beclomethasone, budesonide, fluticasone, ciclesonide, mometasone as well as salts and/or solvates thereof. In some embodiments, the medicament (API) is glycopyrronium bromide, formoterol fumarate, tiotropium bromide In some embodiments, the medicament (API) is a glucocorticosteroid, such as, for example, fluticasone, budesonide, mometasone or ciclesonide.

The term “excipient” is used to describe an ingredient other than the active pharmaceutical ingredients. The selection of an excipient can depend on factors such as the particular mode of administration, the effect of the excipient on solubility and stability, and the nature of the dosage form.

The term “aerosolized medicament” refers to a particulate and/or liquid medicament that is converted into a fine spray or colloidal suspension in air. Aerosolized medicament can be administered to a patient via inhaler devices such as, for example, MDIs or SMIs.

The headings below are not meant to limit the disclosure in any way; embodiments under any one heading may be used in conjunction with embodiments under any other heading.

Reference will now be made in detail to certain embodiments of the invention, examples of which are illustrated in the accompanying drawings. While the invention will be described in conjunction with the illustrated embodiments, it will be understood that they are not intended to limit the invention to those embodiments.

### Smart Spacer

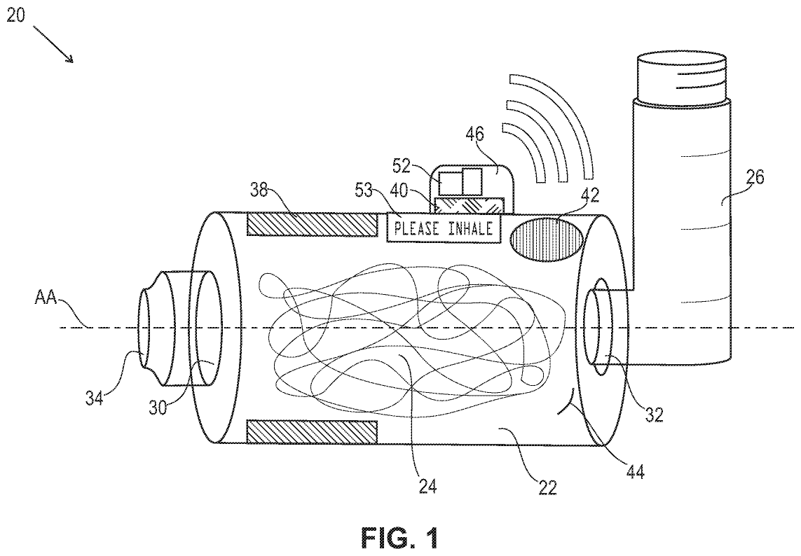

A smart spacer 20, as shown in FIGS. 1-5 is provided that measures and records data collected from at least one sensor such as, for example, a patient's inhaled airflow and exhaled airflow. The smart spacer also generates metrics on inhalation technique, records medicament utilization and generates metrics on lung health. The smart spacer can be used to treat and/or prognose respiratory diseases such as asthma, bronchitis, emphysema and COPD. Patient progress can also be tracked over the period of the smart spacer's use and a historical log can be stored for patient and/or practitioner use.

The smart spacer includes a chamber 22. The chamber is configured to receive and hold an aerosolized medicament 24 dispensed from an inhaler 26, such as a metered-dose inhaler (MDI) or a soft mist inhaler (SMI) within an interior surface 28. The chamber is also configured to receive inhaled air and exhaled air from a patient, as described herein.

In some embodiments, the chamber has dimensions of about 10 to about 20 centimeters (cm) in length and about 3 to about 8 cm in width. In some embodiments, the chamber has a length of about 10, 11, 12, 13, 14, 15, 16, 17, 18, 19 to about 20 cm. In some embodiments, the chamber has a width of about 3, 4, 5, 6, 7 to about 8 cm.

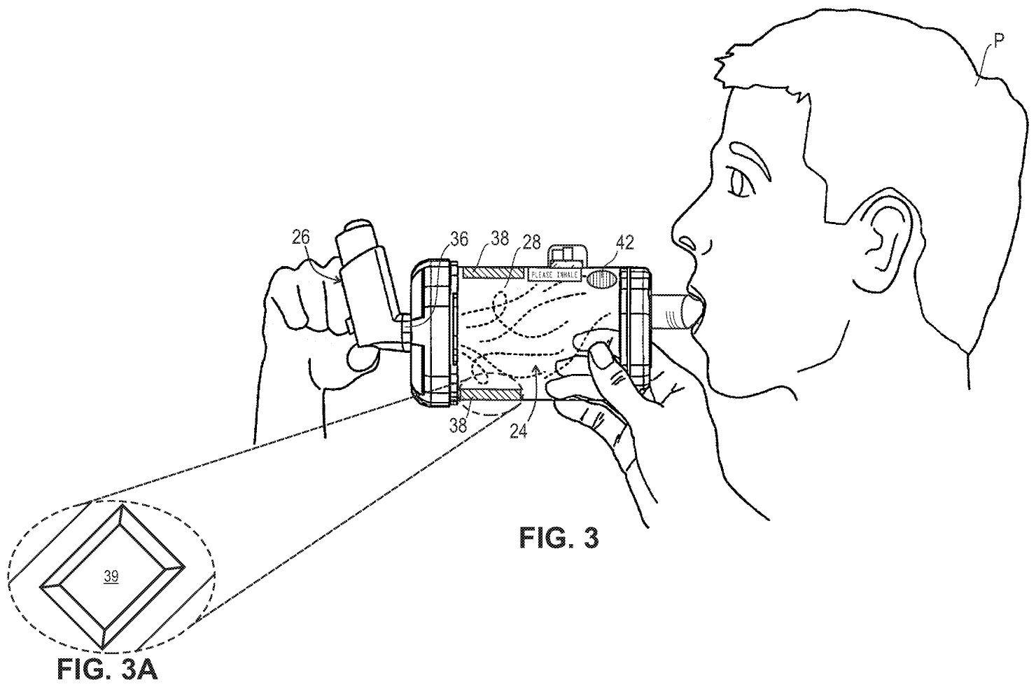

The chamber includes a proximal end opening 30, a distal end opening 32 and a longitudinal axis AA disposed therebetween. The proximal end opening of the chamber includes a mouthpiece 34 or a mask 35 and is configured for engagement with the mouth of the patient P. The mouthpiece is in alignment with the proximal and distal end openings. In some embodiments, the mouthpiece is detachable. The distal end opening is configured for engagement with a portion of the inhaler, such as the mouthpiece 36 of the inhaler, as shown in FIG. 3. The distal end opening can include a collar 37 that facilitates a snug interface between the inhaler and the spacer. The collar can be made from rubber and/or a thermoplastic material. In some embodiments, the mouthpiece of the inhaler and the distal end opening are engaged via an interference fit to allow the mouthpiece of the inhaler to fit snugly in the distal end opening, so that when engaged, the distal end opening will releasably hold the mouthpiece of the inhaler. It will be understood that the proximal end opening and the distal end opening will not interfere with airflow or medicament flow.

In some embodiments, a diameter of the distal end opening is the same or greater than a diameter of the mouthpiece of the inhaler. In some embodiments, the distal end opening has a diameter of about 0.5 cm to about 2 cm and the mouthpiece of the inhaler has a diameter that is about 0.5 cm to about 1.5 cm. In some embodiments, the distal end opening has a length that is the same or greater than a length of the mouthpiece of the inhaler. In some embodiments, the distal end opening has a length of about 1 to about 3 cm and the mouthpiece of the inhaler has a length of about 1 to about 2.5 cm.

In some embodiments, the chamber is devoid of any valve or any one-way valves. In some embodiments, a valve may interfere with medicament delivery to the patient and/or airflow.

The interior surface of the chamber includes at least one sensor 38 for measuring airflow in the chamber. For example, the at least one sensor measures volumetric inhalation airflow and volumetric exhalation airflow of the patient. The at least one sensor is disposed adjacent to the proximal end opening, as shown in FIG. 1. However, it will be understood that the at least one sensor can be placed anywhere on the interior surface of the spacer. For example, the at least one sensor can be disposed adjacent to the distal end opening, or anywhere in between the proximal end opening and the distal end opening. The at least one sensor can be a mass airflow sensor or a differential pressure sensor. The at least one sensor is configured to provide input to a microcontroller 40, as described herein. The at least one sensor is configured to collect, transmit and receive the measurement of airflow and to compile the measurement of airflow as raw airflow data. The airflow data is then inputted or transmitted to the microcontroller from the at least one sensor to compile the airflow data. The data from the at least one sensor can be displayed on the spacer exterior and/or it can be displayed on one or more computers in an easy to read format, such as, for example, a graph, as described herein.

In some embodiments, the at least one sensor can be paired with at least one sensor that measures temperature and barometric pressure to assist in measuring the volumetric inhalation airflow and volumetric exhalation airflow of the patient. In some embodiments, the at least one sensor can be paired with at least one sensor that includes an image sensor, a photodetector sensor, a color detector sensor, a pressure sensor, a temperature sensor, and/or a humidity sensor.

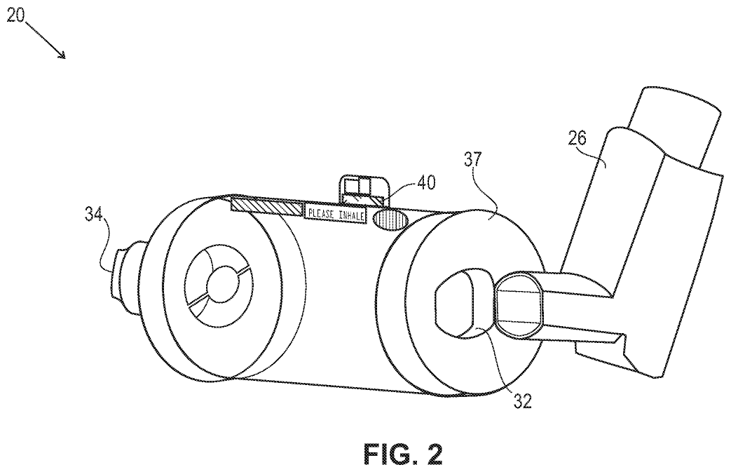

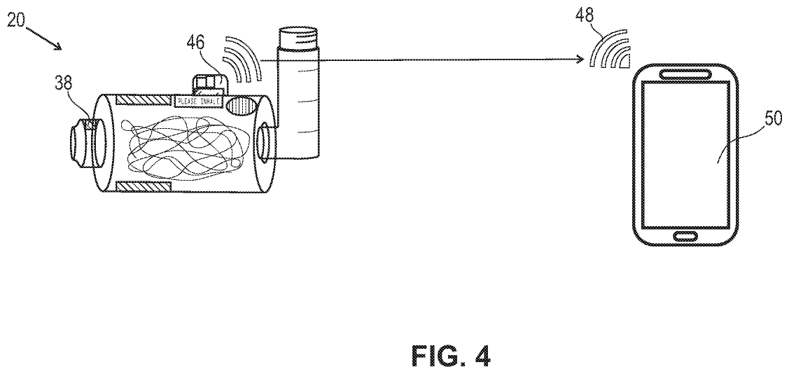

In some embodiments, the at least one sensor comprises a first sensor 38 and a second sensor 38, as shown in FIG. 3. The first sensor and the second sensor can be disposed adjacent to the proximal end opening in a parallel configuration, as shown in FIG. 3. In some embodiments, the at least one sensor can include 1 (FIGS. 2), 2, 3, 4, 5, 6, 7, 8, 9, to 10 sensors disposed within the interior of the chamber. In some embodiments, the at least one sensor is not disposed in or on the mouthpiece of the chamber. In some embodiments, the at least one sensor is disposed in or on the mouthpiece or mask of the chamber, as shown in FIG. 4.

In some embodiments, the at least one sensor can be detachable. In some embodiments, the at least one sensor can be attached to the interior surface of the chamber via adhesive, adhesive strips, Velcro®, clips, hooks, magnets, snaps, buttons, interference fittings, friction fittings, compressive fittings, posts, connectors, and/or fixation plates. In some embodiments, the at least one sensor can be friction fit or snap fit into a recess or an indent 39 located on the interior surface of the chamber, as shown in FIG. 3A. In some embodiments, the shape of the at least one sensor and the shape of the indent are the same. In some embodiments, the length and the width of the indent is slightly greater than the length and the width of the at least one sensor such that the sensor can fixedly engage with the ident. It will be understood, that although the at least one sensor is shown as a rectangular shape, other shapes are contemplated such as, circular, crescent, oval, square, hexagonal, pentagonal, and/or triangular.

In some embodiments, the at least one sensor can be covered by a mesh, such as a hydrophobic mesh such that the one or more sensors are made waterproof. In other embodiments, the at least one sensor can be covered by a plastic sleeve that creates a waterproof environment for the at least one sensor. The at least one sensor can also be coated by hydrophobic films. This will aid in preventing or reducing damage when the spacer and/or the at least one sensor is washed.

The spacer includes an actuator 42 disposed on an exterior surface 44 of the chamber that is configured to initiate measurement of airflow in the chamber. The actuator functions to initiate or turn on the spacer. In some embodiments, the actuator can be a button. The actuator can be disposed anywhere on the exterior surface of the chamber which is easy for the patient to touch. For example, the actuator can be disposed on the exterior surface adjacent to the distal end opening or on a top portion of the exterior surface. In some embodiments, the actuator can be disposed adjacent to the proximal end opening. In some embodiments, the actuator is detachable and can be attached to the exterior surface of the chamber in the same manner as described above with regard to the at least one sensor. The actuator is paired with the microcontroller and is configured to signal the microcontroller to initiate the use of the spacer and to initiate collection of the measurement of airflow. The actuator can be pressed and held (e.g., for about one second or less) to wake the microcontroller from a sleep mode and triggers the at least one sensor to initiate the collection of airflow data. In some embodiments, the actuator can be pressed and held (e.g., for about three seconds or more) to pair a Bluetooth® connection with the spacer and a computer (e.g., patient's personal computer or practitioner's computer), as described herein.

In some embodiments, instead of an actuator (e.g., a button), the spacer can include a motion sensor such that when the patient gently shakes the spacer, the at least one sensor can be activated.

As described above, the spacer includes a microcontroller that is disposed on the exterior surface of the chamber as part of the actuator and/or as part of the at least one sensor. The microcontroller processes the airflow data from the at least one sensor and applies a series of calculations to the airflow data such that airflow metrics are calculated. The measurement of airflow includes measuring peak expiratory flow, forced expiratory volume, forced vital capacity, or a combination thereof. From the measurement of airflow, airflow metrics calculated include volumetric airflow (mL/s), airflow volume (mL), forced expiratory volume in one second (FEV1) and peak expiratory flow (PEF). These measurements can be transmitted, stored and logged into a database to create historical metrics for the patient over time, as well as repeated doses of medicaments.

The microcontroller is powered by a battery and cycles between an active mode and a sleep mode in order to conserve battery life of the microcontroller.

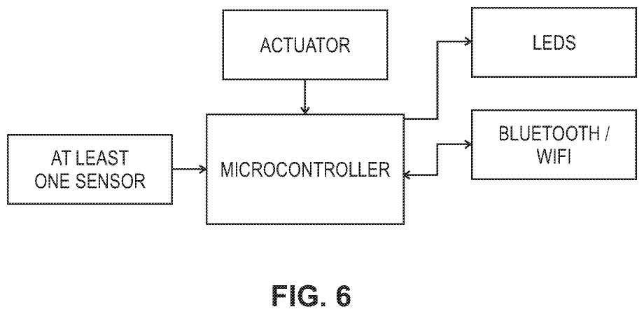

FIG. 6 illustrates a schematic for various inputs/outputs related to spacer use and the microcontroller is shown. Inputs to the microcontroller, include but are not limited to the actuator, the at least one sensor, and/or Bluetooth®/Wi-Fi transmission and data associated therewith. Outputs include, but are not limited to LEDs and/or Bluetooth®/Wi-Fi transmission and data associated therewith.

The spacer includes a radio, such as, for example, a Bluetooth® radio 46 for wireless connection. The Bluetooth® radio is configured to pair with a Bluetooth® radio 48 of a personal computer such as a smartphone 50 or a computer of the patient and/or the practitioner, as described herein. The Bluetooth® radio transmits the airflow data to the smartphone. In some embodiments, the personal computer can be one of a plurality of devices such as, for example, network/stand-alone computers, personal digital assistants (PDAs), WebTV (or other Internet-only) terminals, set-top boxes, cellular/phones, screenphones, pagers, blackberry, smart phones, iPhone, iPad, table, peer/non-peer technologies, kiosks, or other known (wired or wireless) communication devices, etc.

The Bluetooth® radio attempts to establish a wireless connection with the smartphone which serves as the Bluetooth® receiver before streaming the airflow metrics in real time to the smartphone, as shown in FIG. 4. If a valid receiver cannot be found, the microcontroller will indicate that a connection is not present via a status light emitting diode(s) (LED) 52, as described herein. Alternatively, the microcontroller can store the airflow data in memory until a Bluetooth® connection is established and the stored airflow data can be uploaded or downloaded accordingly.

In some embodiments, the microcontroller of the spacer can send the airflow data and/or airflow metrics to a cloud network. The cloud network is a data network environment in which the airflow data and/or airflow metrics from the microcontroller can be stored in a network-attached storage, instead of being solely stored in a local storage.

In some embodiments, the airflow data and/or airflow metrics collected from the spacer (e.g., microcontroller) can be transmitted to a computer, smartphone, or other database and associated with stored medical record data for the particular patient including, among other things, the patient's name, date of birth, sex, address, name of the medicament prescribed, strength, number of days for the MDI or SMI to be used, quantity dispensed, prescriber name, prescription number, pharmacy where filled, number of refills and/or other information.

The spacer includes indicia, such as LEDs, as disclosed above, located on the exterior surface of the chamber. The LEDs indicate when the spacer is awake, when the spacer is connected to a paired smartphone and when pairing between the spacer and the smartphone is occurring. In some embodiments, the indicia can be one or more LED lights used as visual indicators. In some embodiments, the LEDs can be paired with an audio signature such as a buzzer or tune. The indicia may also include unique indicia comprising at least one color, letter, sound, light, and/or video. In some embodiments, the LEDs can be various colors, such as, for example, blue, red, yellow, white, green, purple, pink and/or orange.

In some embodiments, the spacer comprises a display 53, as shown in FIG. 1, on the exterior surface that displays indicia to instruct a patient to inhale, exhale, to aerosolize the medicament or to show measurement of airflow in the chamber from the at least one sensor. For example, in some embodiments in an inhalation mode, as described below, the display will instruct the patient to inhale 1, 2, 3, 4, 5 or 6 times. In some embodiments in an exhalation mode, as described below, the display will instruct the patient to exhale 1, 2, 3, 4, 5 or 6 times. In some embodiments, the display can visually display the airflow data/metrics to the patient in the form of a graph or data points. In some embodiments, the display visually displays reminders to patients to take their medicament, and can provide coaching alerts, relapsing alerts, etc.

The smartphone is loaded with a software program (e.g., smartphone application) that stores the airflow data/metrics and interfaces with the patient such that the airflow data/metrics can be searched, retrieved and displayed by the patient and/or the practitioner. The software program can also be associated with a message digest with a date and time stamp of medicament use that can be a part of a history log of metrics for that patient. The date and time of medicament use is confirmed when the actuator is pressed and when the at least one sensor indicates airflow via patient inhalation. In some embodiments, the airflow data can also be transmitted via Wi-Fi to a web dashboard on a computer 54. The web dashboard generates a report for the medical practitioner.

In some embodiments, the airflow data may be downloaded in one or more textual/graphical formats (e.g., RTF, PDF, TIFF, JPEG, STL, XML, XDFL, TXT etc.), or set for alternative delivery to the smartphone and/or the web dashboard of the computer. The patient may view the airflow data results at a user interface, which allows viewing on the same display, such as the screen 56 or monitor 58 of the smartphone and/or the computer.

In some embodiments, the patient and/or the practitioner can interface with the computer (e.g., smartphone, a computer of the practitioner etc.) via a user interface that may include one or more display devices (e.g., CRT, LCD, or other known displays) or other output devices (e.g., printer, etc.), and one or more input devices (e.g., keyboard, mouse, stylus, touch screen interface, or other known input mechanisms) for facilitating interaction of the patient and/or the practitioner with the airflow data from the smart spacer via the user interface. The user interface may be directly coupled to an airflow database or directly coupled to a network server system via the Internet or cloud computing.

In some embodiments, the user interface device may be implemented as a graphical user interface (GUI) containing a display or the like, or may be a link to other user input/output devices known in the art. Individual or of a plurality of devices (e.g., network/stand-alone computers, personal digital assistants (PDAs), WebTV (or other Internet-only) terminals, set-top boxes, cellular/phones, screenphones, pagers, blackberry, smart phones, iPhone, iPad, table, peer/non-peer technologies, kiosks, or other known (wired or wireless) communication devices, etc.) may similarly be used to execute one or more computer programs (e.g., universal Internet browser programs, dedicated interface programs, etc.) to allow patients to interface with the airflow data in the manner described. Database hardware and software can be developed for access by patients and/or practitioners through personal computers, mainframes, and other processor-based devices. Patients and/or practitioners may access the data stored locally on hard drives, CD-ROMs, stored on network storage devices through a local area network, or stored on remote database systems through one or more disparate network paths (e.g., the Internet).

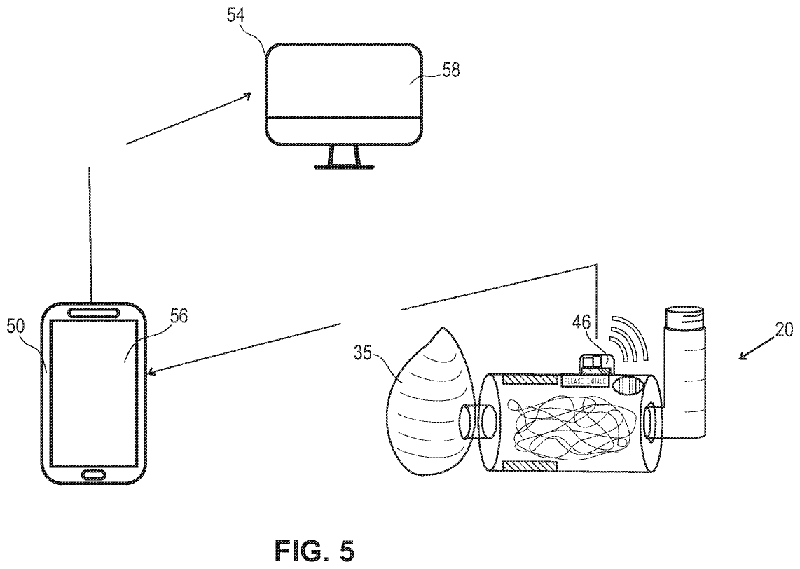

FIG. 5 illustrates a communication loop between the spacer, smartphone and web dashboard of a medical practitioner. Communication between the spacer to the smartphone occurs through Bluetooth® transmission to the smartphone, which then in turn communicates via Wi-Fi/cellular data with the web dashboard of a practitioner.

In some embodiments, the electronic circuitry in the spacer, may include some or all of the capabilities of a computer (e.g., the microcontroller) in communication with a network and/or directly with other computers. The computer may include a processor, a storage device, a display or other output device, an input device, and a network interface device, all connected via a bus. A battery can be provided to couple and power the computer. The computer may communicate with a network. The processor represents a central processing unit of any type of architecture, such as a CISC (Complex Instruction Set Computing), RISC (Reduced Instruction Set Computing), VLIW (Very Long Instruction Word), or a hybrid architecture, although any appropriate processor may be used. The processor executes instructions and includes that portion of the computer that controls the operation of the entire computer. The processor typically includes a control unit that organizes data and program storage in memory and transfers data and other information between the various parts of the computer. The processor receives input data from the input device (e.g., the at least one sensor) and the network reads and stores instructions (for example processor executable code) and data in a main memory, such as random access memory (RAM), static memory, such as read only memory (ROM), and a storage device. The processor may present data to a user via an output device or user interface, as described above, such as the screen of the smartphone or the monitor of the web dashboard of the practitioner's computer or on the display that is located on the spacer.

In some embodiments, the spacer is reusable and washable. The spacer and its components can be made from various materials, such as, for example, plastic, such as a thermoplastic material. In some embodiments, materials include, but are not limited to thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), polyurethane, silicone-polyurethane copolymers, polymeric rubbers, polyolefin rubbers, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy and their combinations.

In some embodiments, the spacer and its components can be sterilized by radiation via terminal sterilization. Terminal sterilization of a product provides greater assurance of sterility than from processes such as an aseptic process, which requires individual product components to be sterilized separately and the final package assembled in a sterile environment.

In various embodiments, gamma radiation is used in the terminal sterilization step, which involves utilizing ionizing energy. Gamma rays are highly effective in killing microorganisms, they leave no residues, nor do they have sufficient energy to impart radioactivity to the spacer. Gamma rays can be employed when the spacer is in a package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the spacer. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

### Spacer Inhalation and Exhalations Modes

The smart spacer of the current application can provide at least three functions into a single device. These functions include, medicament utilization tracking, inhalation technique coaching and lung health tracking, as described below. The spacer includes an inhalation mode and an exhalation mode to perform these functions. The inhalation mode is associated with utilization tracking and inhalation technique coaching and the exhalation mode functions as a spirometer, as described below which is associated with the function of lung health tracking.

Inhalation Mode: Medicament Utilization Tracking

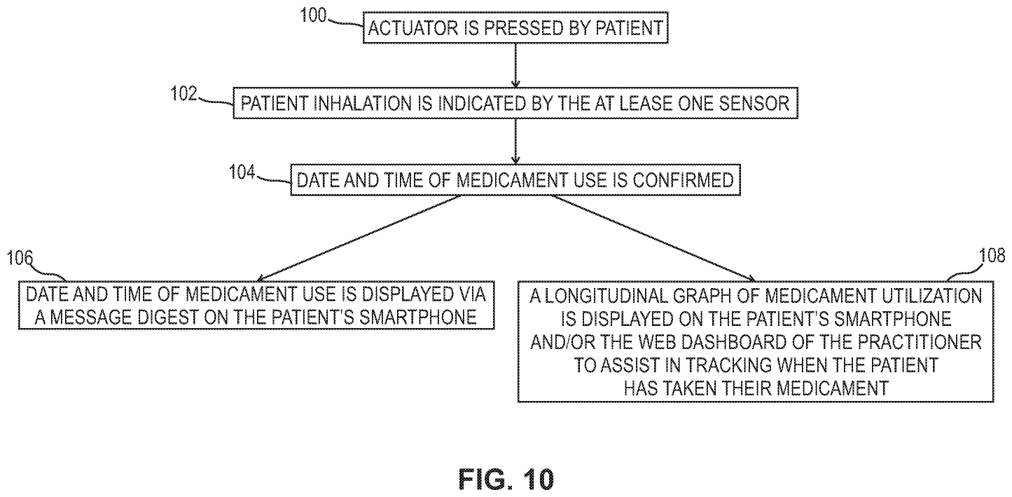

In the inhalation mode for medicament utilization tracking, as shown in FIG. 10, a medicament utilization event is logged whenever the patient presses on the actuator 100 on the spacer and patient inhalation is indicated/recorded by the at least one sensor 102. The date and time of medicament use is confirmed 104 when the actuator is pressed and when the at least one sensor indicates airflow via patient inhalation. The date and time of medicament use is then displayed via a message digest on the patient's smartphone 106 or can be stored on the device. A longitudinal graph of medicament utilization is also displayed on the patient's smartphone and/or the web dashboard of the practitioner 108 to assist in tracking when the patient has taken their medicament.

Inhalation Mode: Inhalation Technique Coaching

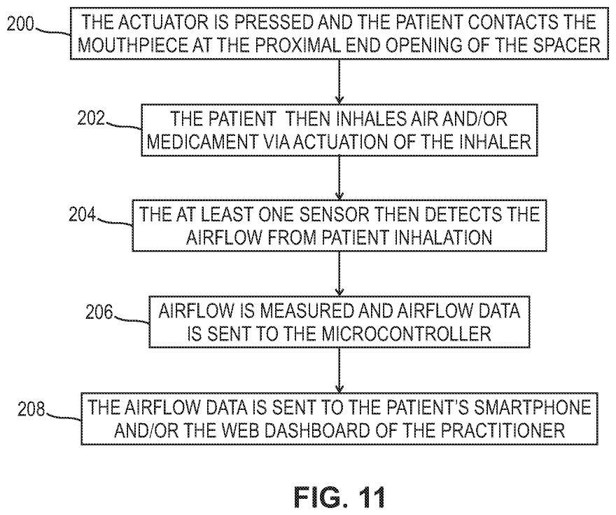

The inhalation technique coaching function indicates whether a patient is breathing too shallow and can assist the patient in learning how to breathe deeply and slowly. In operation, as shown by the flowchart of FIG. 11, the actuator is pressed, and the patient contacts the mouthpiece at the proximal end opening of the spacer 200. In this mode, the inhaler engages with the distal end opening of the spacer. The patient then inhales air and/or medicament via actuation of the inhaler 202. The at least one sensor then detects the airflow from patient inhalation 204. Airflow is measured and airflow data is sent to the microcontroller 206. From there, the airflow data is sent to the patient's smartphone and/or the web dashboard of the practitioner 208.

The spacer is used to track the inhalation technique of the patient such that when the at least one sensor detects the direction of airflow, volumetric flow rate is measured throughout the duration of inhalation. From airflow data compiled, the microcontroller calculates the total inhaled volume and duration which is then used guide the patient on inhalation technique through an application of a personal computer, such as a smartphone. The practitioner can also view these results via a web dashboard, as described above.

In some embodiments, the application on the personal computer or the display on the spacer can also display phrases that show patient progress or regression. For example, the phrases can include, but are not limited to “good job,” “please inhale deeper,” “please slow down,” and/or “please stop.” Longitudinal graphs on patient progress can also be displayed.

In addition to the airflow data, inhalation technique coaching also factors in the body weight and height of the patient, title volume (TV), and inhalation time.

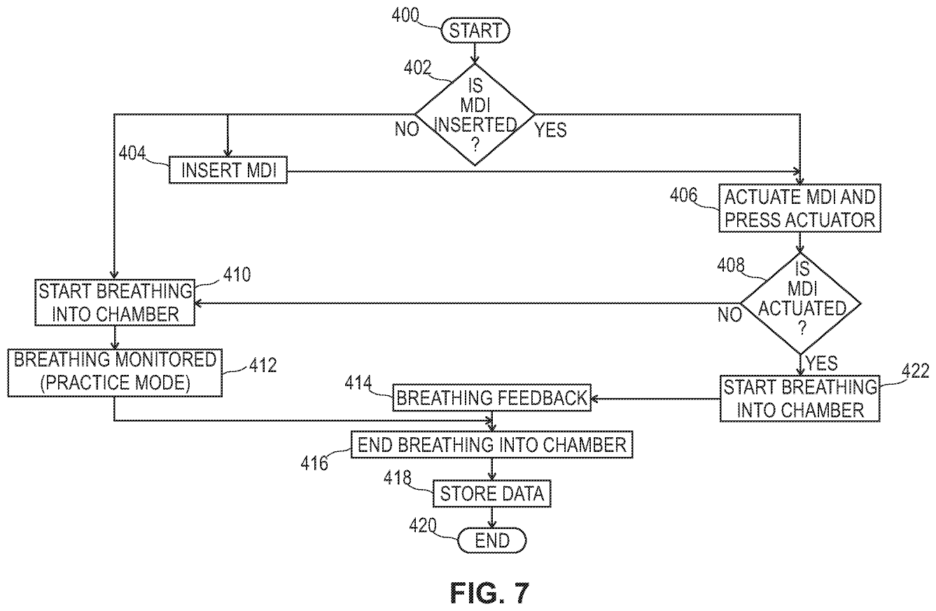

FIG. 7 is a flow chart illustrating inhaler usage and feedback loops of the spacer when the spacer is in the inhalation mode for medicament utilization tracking. For example, to start/initiate use of the spacer 400, the query “is MDI inserted” 402 is asked. If no, then the user must insert the inhaler (MDI) 404. If yes, then the MDI is actuated 406 and the actuator is pressed. The query “is MDI actuated?” 408 is asked and if no, then the patient starts breathing into the chamber 410. In some embodiments, the breathing is then monitored as a practice mode 412. Breathing feedback is then provided 414, breathing is ended in the chamber 416, the data is stored 418 and the use of the spacer ends 420. If yes, then the patient starts to breathe into the chamber 422 and breathing feedback is provided 414. The patient then ends breathing into the chamber 416, the data is stored 418 and the use of the spacer ends 420. In this embodiment, the data stored can be the date and time of inhalation for medicament utilization tracking.

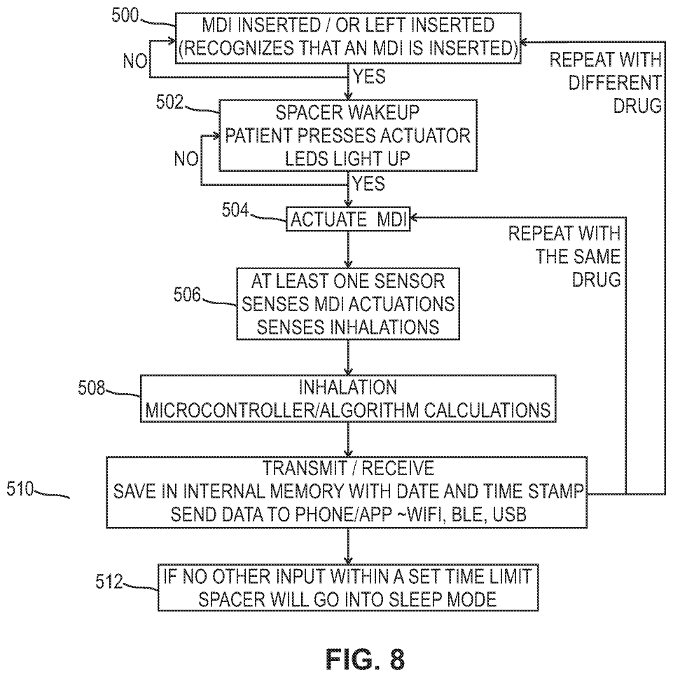

FIG. 8 is a flow chart illustrating inhaler usage and feedback loops of the spacer in the inhalation mode for inhalation technique coaching. For example, the inhaler (MDI) is inserted or is left inserted into the spacer 500. In some embodiments, a sensor can be provided at the distal end opening of the chamber such that when the inhaler engages the distal end opening, feedback can be provided to indicate that the inhaler is inserted. If no, then the MDI is inserted into the distal end opening of the chamber. If yes, then the spacer “wakes up” 502 via the patient pressing the actuator. The LEDs will light up to indicate that the spacer is awake. If no, then the actuator is pressed again to wake up the spacer. If yes, then the MDI is actuated 504. The at least one sensor is then actuated 506 and inhalation is detected 508.

The at least one sensor detects airflow via its direction, and volumetric flow rate is measured throughout the duration of the inhalation. In some embodiments, airflow data measured by the at least one sensor is sent to the microcontroller such that the microcontroller can store data and can implement calculations for determining airflow metrics. The data is then transmitted and received 510. The data is saved in internal memory of the microcontroller with a date and time stamp. The data is then sent to the patient's smartphone and/or the web dashboard of the practitioner's computer. The data is used to guide the patient on their inhaler technique via the patient's smartphone loaded with an application (e.g., did the patient breathe slow enough and did they breathe deep enough). The MDI can be actuated again 504, and the steps repeated. Further, after the data is transmitted and received, the patient can repeat these steps and go back to inserting a different inhaler (MDI) 500. If no other input occurs, then the spacer will go into a sleep mode 512.

Exhalation Mode: Lung Health Tracking

The spacer can be used in the exhalation mode as a spirometer. This will be where the inhaler has been removed from the spacer and, in some embodiments, the distal end opening remains open and is unobstructed by the inhaler. In some embodiments, the spacer as a spirometer can perform a pulmonary function test (PFT) which measures lung capacities of patients. A spirometer measures values of forced vital capacity (FVC), FEV1, PEF, flow volume ring, maximum voluntary ventilation (MVV), maximal inspiratory/expiratory pressures (MIP/MEP), airway resistance (Raw) and compliance (C), functional residual capacity (FRC), total lung capacity (TLC), residual volume (RV) FRC/TLC ratio, minute ventilation, alveolar ventilation and dead space, ventilation distribution, and diffusion capacity upon the patient performing different breathing maneuvers.

In PFT, as the patient exhales air into the spacer acting as a spirometer, the volume, intensity, and flow of air at different times will be measured by the at least one sensor. Parameters of FVC and forced expiratory volume in one second are mostly used in dynamic ventilation tests and can be performed by using the spacer in the spirometry mode. In the mentioned tests, the volume of air exhaled from the lungs by breathing out enforcedly, quickly and deeply is measured. The volume of air expired by the patient in the first second is measured by FEV1 value. The FEV value can be used in volume-time graphs. The patient's volume of expiration by time can be visualized by means of the volume-time graphs and it is examined by making comparison where it is located in the required curve. Also, a flow-time curve is examined in PFT. A volume-time curve is created by measuring the expiration of the patient and the flow generated within the spacer. The flow-volume curve is used for an interpretation of the factors affecting the flow occurring in airways. The top of the flow-volume curve indicates the maximum expiratory flow rate (peak expiratory flow (PEF). PEF value indicates diameter of large airways and activity of respiratory muscles. Evaluation and interpretation of PFT result are performed through the comparison of the measured values to the expected values. The expected values used in test evaluation are obtained as a result of studies carried out with healthy persons of different age, gender, height, weight, and race groups.

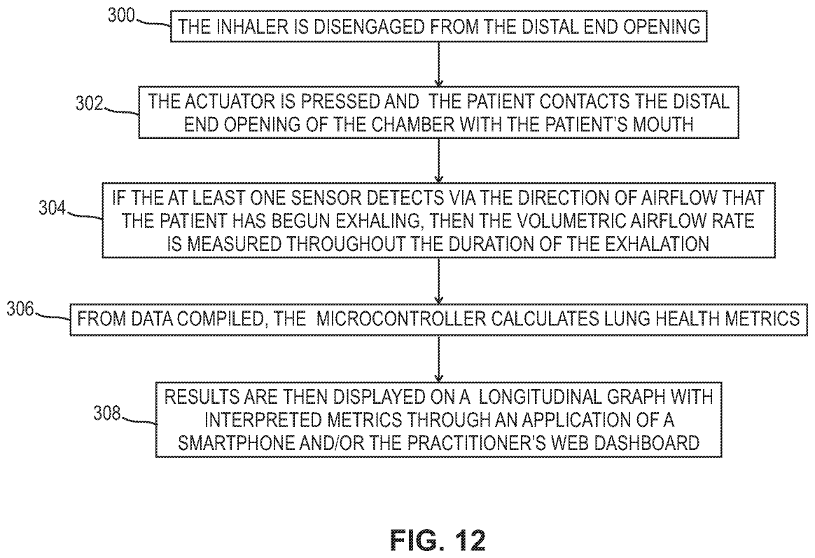

FIG. 12 is a flow chart of the spacer exhalation mode or spirometry mode. In the exhalation mode, the inhaler is disengaged from the distal end opening 300, as shown in FIG. 2 such that the peak flow is measured in the opposite direction of the spacer. The actuator is pressed (to wake the microcontroller and the at least one sensor, and to turn on the status LED) and the patient contacts the distal end opening of the chamber with the patient's mouth 302. In some embodiments, the patient contacts the proximal end opening with the patient's mouth. In some embodiments, the display will instruct the patient to exhale 1, 2, 3, 4, 5 or 6 times.

If the at least one sensor detects via the direction of airflow that the patient has begun exhaling, then the volumetric airflow rate is measured throughout the duration of the exhalation 304. From data compiled, the microcontroller of the spacer calculates airflow metrics or lung health metrics 306 such as forced expiratory volume within the first second of exhalation (FEV1) and peak expiratory flow (PEF). The results are then displayed on a longitudinal graph with interpreted metrics through an application of a personal computer, such as a smartphone and/or the practitioner can view the results via a web dashboard 308.

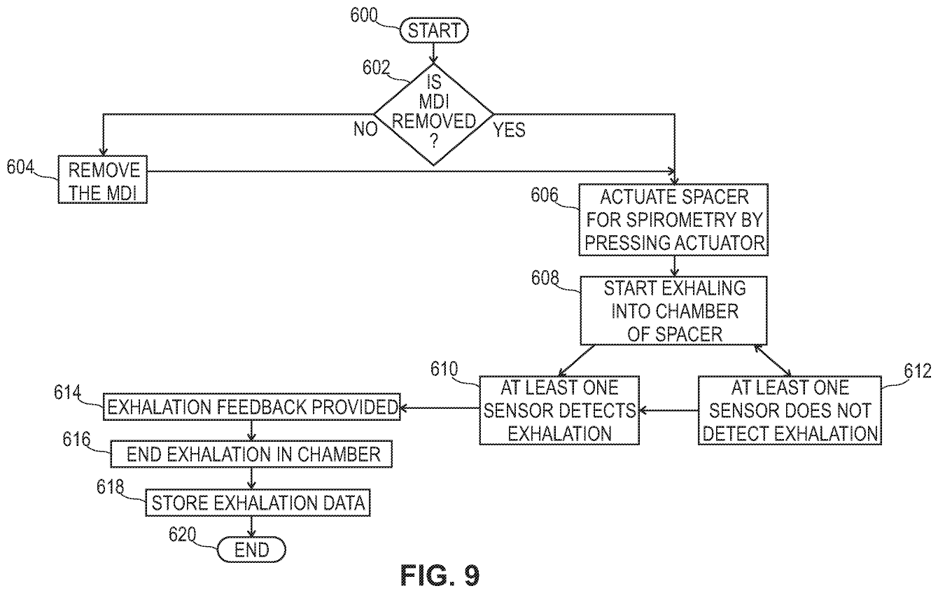

FIG. 9 is a flow chart illustrating inhaler usage and feedback loops of the spacer when the spacer is in the exhalation mode or spirometry mode. For example, to start/initiate use of the spacer 600, the query “is MDI removed” 602 is asked. If no, then the user must remove the inhaler (MDI) 604. If yes, then the spacer is actuated for spirometry 606 by pressing the actuator. The patient is then instructed to exhale into chamber of spacer 608. If the at least one sensor detects exhalation 610, then the patient has begun exhaling. If the at least one sensor does not detect exhalation 612, then the patient is instructed to exhale. Exhalation feedback is then provided 614, exhalation is ended in the chamber 616, the exhalation data is stored 618 and the use of the spacer in spirometry mode ends 620.

### Medicaments (APIs)

In some embodiments, the medicament (API) can be dispensed from an inhaler such as a metered-dose inhaler (MDI) or a soft mist inhaler (SMI). The medicament (API) can be any medicament suitable for inhalation including an anticholinergic agent, a bronchodilator, and/or a corticosteroids.

The anticholinergic agents include, but are not limited to trihexyphenidyl, benztropine mesylate, ipratropium, tiotropium, orphenadrine, atropine, flavoxate, oxybutynin, scopolamine, hyoscyamine, tolterodine, belladonna alkaloids, fesoterodine, solifenacin, darifenacin, propantheline, 5-[3-(3-Hydroxyphenoxy)azetidin-1-yl]-5-methyl-2,2-diphenylhexanamide or a combination thereof. The bronchodilators include, but are not limited to adrenergic bronchodilators including, but not limited to, levalbuterol, metaproterenol, pirbuterol, formoterol, terbutaline, or albuterol; anticholinergic bronchodilators including, but not limited to, aclidinium systemic, ipratropium systemic, tiotropium systemic, orumeclidinium systemic; bronchodilator combinations including, but not limited to, umeclidinium/vilanterol systemic, budesonide/formoterol systemic, fluticasone/salmeterol systemic, albuterol/ipratropium systemic, fluticasone/vilanterol systemic, olodaterol/tiotropium systemic, formoterol/mometasone systemic, formoterol/glycopyrrolate systemic, orglycopyrrolate/indacaterol systemic; and methylxanthines including, but not limited to, theophylline systemic, aminophylline systemic, or dyphylline systemic. The corticosteroids include, but are not limited to, beclomethasone, budesonide, flunisolide, fluticasone, mometasone, ciclesonide or tiotropium.

The medicament (API) may include the medicaments (APIs) described above, and may also include substances traditionally regarded as actives, drugs and bioactive agents, as well as biopharmaceuticals typically employed to treat a number of conditions which is defined broadly to encompass diseases, disorders, infections, and the like. Exemplary medicaments (APIs), without limitation, antibiotics, antivirals, H2-receptor antagonists, 5HT1 agonists, 5HT3 antagonists, and COX2-inhibitors.

In some embodiments, the medicaments (APIs) may also be selected from, analgesics, e.g., codeine, dihydromorphine, ergotamine, fentanyl or morphine; anginal preparations, e.g., diltiazem; antiallergics, e.g., cromoglycate (e.g. as the sodium salt), ketoprofen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, prednisone, prednisolone, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl)ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline. It will be clear to a person skilled in the art that, where appropriate, the medicaments (APIs) may be used in the form of salts, (e.g., as alkali metal or amine salts or as acid addition salts) or as esters (e.g., lower alkyl esters) or as solvates (e.g., hydrates) to optimize the activity and/or stability of the medicament.

Additionally, the medicaments (APIs) may be selected from, for example, antibiotics. Such antibiotics include, for example, nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. In the following examples of such antibiotics are listed: ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth sub salicylate, colloidal bismuth subcitrate, gramicidin, mecillinam, cloxiquine, chlorhexidine, dichlorobenzylalcohol, methyl-2-pentylphenol. The active antibiotics could be in standard forms or used as salts, hydrates, esters etc. A combination of two or more of the above listed drugs may be used. The antibiotics can be clarithromycin, erythromycin, roxithromycin, azithromycin, amoxicillin, metronidazole, tinidazole and tetracycline. Clarithromycin and metronidazole alone or in combination are especially suitable.

Moreover, medicaments (APIs) may also be selected from, for example, antivirals. Examples of APIs that are effective for the treatment of viral and viral associated conditions are (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine(oxetanocin-G), acyclic nucleosides, for example acyclovir, valaciclovir, famciclovir, ganciclovir, and penciclovir, acyclic nucleoside phosphonates, for example (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid(tenofovir), and (R)-[[2-(6-Amino-9H-purin-9-yl)-1-methylethoxy]methyl]phosphonic acid bis-(isopropoxy carbonyloxymethyl)ester (bis-POC-PMPA), ribonucleotide reductase inhibitors, for example 2-acetylpyridine 5-[(2-chloroanilino)thiocarbonyl)thiocarbonohydrazone and hydroxyurea, nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurinedioxolane (DAPD), 3′-azido-2′,3′-dideoxythymidine-5′-H-phosphophonate (phosphonovir), 2′-deoxy-5-iodo-uridine(idoxuridine), (−)-cis-1-(2-hydroxymethyl)-1,3-oxathiolane 5-yl)-cytosine(lamivudine), cis-1-(2-(hydroxymethyl)-1,3-oxathiolan-5-yl)-5-fluorocytosine(FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol(abacavir), 9-[4-hydroxy-2-(hydroxymethyl)but-1-yl]guanine(H2G), ABT-606 (2HM-H2G) and ribavirin, protease inhibitors, for example indinavir, ritonavir, nelfinavir, amprenavir, saquinavir, (R)—N-tert-butyl-3-[(2S,3S)-2-hydroxy-3-N—[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha, 5 alpha,6beta)]-1,3-bis[(3-aminophenyl)methyl]hexahydro-5,6-dihydroxy-4,7-bis(phenylmethyl)-2H-1,3-diazepin-2-one dimethanesulfonate(mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone(tipranavir), N′-[2(S)-Hydroxy-3 (S)—[N-(methoxycarbonyl)-1-tert-leucylamino]-4-phenylbutyl-Nalpha-(methoxycarbonyl)-N′-[4-(2-pyridyl)benzyl]-L-tert-leucylhydrazide(BMS-232632), 3-(2(S)-Hydroxy-3 (S)-(3-hydroxy-2-methylbenzamido)-4-phenylbutanoyl)-5,5-dimethyl-N-(2-methylbenzyl)thiazolidine-4(R)-carboxamide(AG-1776), N-(2(R)-hydroxy-1(S)-indanyl)-2(R)-phenyl-methyl-4(S)-hydroxy-5-(1-(1-(4-benzo[b]furanylmethyl)-2(S)—N′-(tert-butylcarboxamido)piperazinyl)pentanamide (MK-944A), and (3S)-tetrahydrofuran-3-yl (1S,2R)-[[(4-aminophenyl)sulphonyl)](isobutyl)amino]-1-benzyl-2-(phosphonooxy)propylcarbamate monocalcium salt(fosamprenavir), interferons such as α-interferon or a combination thereof.

The medicament may also include pharmaceutically acceptable salts, esters, solvates, and/or hydrates of the pharmaceutically active substances referred to hereinabove. Various combinations of any of the above medicaments may also be employed.

The medicament further comprises an excipient and can comprise glucose, arabinose, lactose, sucrose, maltose, mannitol, dextrans, magnesium stearate, leucine, isoleucine, lysine, valine, methionine, phenylalanine, or a combination thereof. In some embodiments, the lactose is lactose monohydrate. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; and mixtures thereof. In some embodiments, the at least first layer and/or the at least second layer can comprise more than one excipient.

In some embodiments, the inhaler does not require a propellant. However, in some embodiments, the inhaler can contain propellants, including, but not limited to hydrofluoroalkane (HFA), such as chlorodifluoromethan, trifluoromonofluoroethane, chlorodifluoroethane, difluoroethane, heptafluoropropane or a combination thereof.

### Methods of Making

The present disclosure also provides methods of making a spacer, the method comprising providing a spacer comprising a chamber having an interior surface, an exterior surface, and a proximal opening and a distal opening opposite the proximal opening, the interior surface of the chamber configured to receive inhalation air and exhalation air from a patient, attaching to the interior surface of the chamber at least one sensor for measuring airflow in the chamber; and attaching an actuator on the exterior surface of the chamber, the actuator configured to initiate measurement of airflow in the chamber. It is to be understood that the spacer described is the spacer of FIGS. 1-5.

In some embodiments, a method of using a spacer is provided. The method comprises attaching an inhaler to a distal end opening of the spacer, the spacer comprising a chamber having an interior surface and an exterior surface, the interior surface of the chamber configured to receive aerosolized medicament from an inhaler and having at least one sensor for measuring airflow in the chamber; and an actuator disposed on the exterior surface of the chamber configured to initiate measurement of airflow in the chamber. It is to be understood that the spacer described is the spacer of FIGS. 1-5.

In some embodiments, the spacer comprises a display on the exterior surface that displays indicia to instruct a patient to inhale, exhale, or aerosolize the medicament. In some embodiments, the method further comprises contacting the actuator to initiate the at least one sensor to collect airflow data and contacting the inhaler to aerosolize the medicament in the chamber.

In some embodiments, a method of using a spacer is provided. The method comprises removing an inhaler from a distal end opening of the spacer, the spacer having a chamber having an interior surface and an exterior surface, the interior surface of the chamber configured to receive inhalation air and exhalation air from a patient, the interior surface having at least one sensor for measuring airflow in the chamber; and an actuator disposed on the exterior surface of the chamber configured to initiate measurement of airflow in the chamber. It is to be understood that the spacer described is the spacer of FIGS. 1-5. Further, it is to be understood that the current method uses the spacer as a spirometer.

In some embodiments, the spacer comprises a display on the exterior surface that displays indicia to instruct a patient to inhale, exhale, or show measurement of airflow in the chamber from the at least one sensor. In some embodiments, the method further comprises contacting the actuator to initiate the at least one sensor to collect airflow data and causing the display to display indicia to instruct the patient to inhale or exhale.

In some embodiments, the smart spacer can be used in a method of treating, diagnosing or prognosing the following diseases, asthma, bronchitis, emphysema and/or COPD.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A spacer comprising a chamber having an interior surface and an exterior surface, the interior surface of the chamber configured to receive aerosolized medicament from an inhaler and having at least one sensor for measuring airflow in the chamber; and an actuator disposed on the exterior surface of the chamber configured to initiate measurement of airflow in the chamber.

2. The spacer of claim 1, wherein the interior surface of the chamber is configured to hold aerosolized medicament from the inhaler in the chamber.

3. The spacer of claim 1, wherein the interior surface of the chamber is configured to receive inhaled air and exhaled air from a patient.

4. The spacer of claim 1, wherein the inhaler is a metered dose inhaler (MDI) or a soft mist inhaler (SMI).

5. A spacer comprising a chamber having an interior surface and an exterior surface, the interior surface of the chamber configured to receive inhalation air and exhalation air from a patient, the interior surface having at least one sensor for measuring airflow in the chamber; and an actuator disposed on the exterior surface of the chamber configured to initiate measurement of airflow in the chamber.

6. The spacer of claim 5, wherein the chamber comprises a proximal end opening and a distal end opening, and the at least one sensor is disposed adjacent to the proximal end opening.

7. The spacer of claim 6, wherein the proximal end opening comprises a mouthpiece or a mask and the mouthpiece or the mask is configured for engagement with a mouth of the patient, and the distal end opening is configured for engagement with a mouthpiece of the inhaler.

8. The spacer of claim 7, wherein the at least one sensor is not disposed in or on the mouthpiece.

9. The spacer of claim 5, wherein the measurement of airflow includes measuring peak expiratory flow, forced expiratory volume, forced vital capacity, or a combination thereof

10. The spacer of claim 5, wherein the spacer has an inhalation mode and an exhalation mode.

11. The spacer of claim 10, wherein the exhalation mode functions as a spirometer.

12. The spacer of claim 5, wherein the actuator is paired with a microcontroller, the actuator configured to signal the microcontroller to initiate the use of the spacer and to initiate collection of the measurement of airflow.

13. The spacer of claim 12, wherein the at least one sensor is configured to collect, transmit and receive the measurement of airflow and to compile the measurement of airflow as airflow data.

14. The spacer of claim 13, wherein the airflow data is transmitted to the microcontroller from the at least one sensor to compile the airflow data.

15. The spacer of claim 12, wherein microcontroller is disposed on the exterior surface of the chamber.

16. The spacer of claim 5, wherein the actuator is detachable.

17. The spacer of claim 5, wherein the at least one sensor comprises a first sensor and a second sensor.

18. The spacer of claim 5, wherein the at least one sensor is detachable.

19. The spacer of claim 14, wherein the spacer comprises a Bluetooth® radio for wireless connection, the Bluetooth® radio configured to pair with a Bluetooth® radio of a personal computer comprising a smartphone.

20. The spacer of claim 19, wherein the Bluetooth® radio transmits the airflow data to the smartphone.

21. The spacer of claim 20, wherein the smartphone is loaded with a software program that stores the airflow data and interfaces with the patient such that the airflow data is delivered to the patient.

22. The spacer of claim 21, wherein the airflow data can be transmitted via Wi-Fi to a web dashboard on a computer.

23. The spacer of claim 5, wherein the spacer comprises indicia on the exterior surface of the chamber, the indicia comprising one or more light emitting diodes (LED).

24. The spacer of claim 1, wherein the spacer is reusable and washable.

25. A method of making a spacer, the method comprising providing a spacer comprising a chamber having an interior surface, an exterior surface, and a proximal opening and a distal opening opposite the proximal opening, the interior surface of the chamber configured to receive inhalation air and exhalation air from a patient, attaching to the interior surface of the chamber at least one sensor for measuring airflow in the chamber; and attaching an actuator on the exterior surface of the chamber, the actuator configured to initiate measurement of airflow in the chamber.

26. A method of using a spacer, the method comprising attaching an inhaler to a distal end opening of the spacer, the spacer comprising a chamber having an interior surface and an exterior surface, the interior surface of the chamber configured to receive aerosolized medicament from an inhaler and having at least one sensor for measuring airflow in the chamber; and an actuator disposed on the exterior surface of the chamber configured to initiate measurement of airflow in the chamber.

27. The method of claim 26, wherein the spacer comprises a display on the exterior surface that displays indicia to instruct a patient to inhale, exhale, or aerosolize the medicament.

28. The method of claim 26, further comprising contacting the actuator to initiate the at least one sensor to collect airflow data and contacting the inhaler to aerosolize the medicament in the chamber.

29. A method of using a spacer, the method comprising removing an inhaler from a distal end opening of the spacer, the spacer having a chamber having an interior surface and an exterior surface, the interior surface of the chamber configured to receive inhalation air and exhalation air from a patient, the interior surface having at least one sensor for measuring airflow in the chamber; and an actuator disposed on the exterior surface of the chamber configured to initiate measurement of airflow in the chamber.

30. The method of claim 29, wherein the spacer comprises a display on the exterior surface that displays indicia to instruct a patient to inhale, exhale, or show measurement of airflow in the chamber from the at least one sensor.

31. The method of claim 29, further comprising contacting the actuator to initiate the at least one sensor to collect airflow data and causing the display to display indicia to instruct the patient to inhale or exhale.

32. A spacer comprising a chamber comprising a proximal end opening, a distal end opening, an exterior surface and an interior surface, the proximal end opening comprising a mouthpiece, and the interior surface of the chamber configured to receive inhalation air and exhalation air from a patient, the mouthpiece having at least one sensor for measuring airflow in the chamber; and an actuator disposed on the exterior surface of the chamber configured to initiate measurement of airflow in the chamber.

33. The spacer of claim 32, wherein the airflow measured is airflow data that can be transmitted via Wi-Fi to a web dashboard on a computer.
